(19) United States
(12) Patent Application Publication
(10) Pub. No.: US 2018/0096741 A1
(43) Pub. Date: Apr. 5, 2018

(54) DEVICE FOR AUTOMATICALLY SELECTING KEY MEDICAL IMAGE

(71) Applicant: TPLUS, SeouL (KR)

(72) Inventors: Jungho LEE, Seoul (KR); Dongha PARK, Namyangju-si, Gyeonggi-do (KR); Youngtae SEO, Seoul (KR)

(73) Assignee: TPLUS, Seoul (KR)

(21) Appl. No.: 15/560,163

(22) PCT Filed: Apr. 26, 2016
(86) PCT No.: PCT/KR2016/004326

§ 371 Date: Sep. 21, 2017

(30) Foreign Application Priority Data
Apr. 30, 2015 (KR) 10-2015-0061734
Feb. 12, 2016 (KR) 10-2016-0016054

(51) Int. Cl. G16H 30/20; A61B 6/03

(52) U.S. Cl. CPC G16H 30/20; A61B 6/465; A61B 6/032

(57) ABSTRACT

Disclosed is a device for automatically selecting key medical images. A device for generating a key medical image according to an aspect of the invention may include: an input check module configured to identify a clinical image for which a record was inputted from among multiple clinical images; a display check module configured to identify a clinical image which has been displayed from among the multiple clinical images; a time check module configured to identify a clinical image which has been displayed for a particular duration of time or longer from among the multiple clinical images; and a key image registry module configured to assign points to clinical images identified by the input check module, the display check module, and the time check module and register a clinical image having a certain amount of points or more assigned thereto as a key image.

### BACKGROUND

### Technical Field

The present invention relates to a device for automatically selecting key medical images, more particularly to a device for automatically selecting key images from multiple medical images.

### Description of the Related Art

For the purpose of diagnosis, etc., hospitals utilize medical imaging devices (modality) such as X-ray, CT (computed tomography), and PET (positron emission topography) devices. An imaging device would photograph a patient's body to generate several clinical images, and such clinical images may be converted into a DICOM (Digital Imaging and Communications in Medicine) file before they are provided to the medical staff by a PACS (picture archiving and communication system).

In a clinical image information exchange system, the clinical images taken at one hospital would have to be transmitted to another hospital for viewing, and in most of these cases, the entirety of the clinical images have to be provided, not just the required few. In the case of an imaging device such as a CT device, etc., one round of scanning can generate tens to hundreds of clinical images, and since such clinical images have very large sizes, transmitting all of these images may be very difficult and may require a large amount of time.

### SUMMARY OF THE INVENTION

An aspect of the present invention, conceived to resolve the problem described above, is to provide a device for automatically selecting key medical images that can generate key images automatically from clinical images to obviate the need to transmit unnecessary clinical images.

Other objectives of the present invention will be more clearly understood from the embodiments of the invention set forth below.

### Technical Solution

A device for generating a key medical image according to an aspect of the invention may extract a clinical image for which a record was inputted by a user, from among multiple clinical images, and register the extracted clinical image as a key image.

A device for generating a key medical image according to another aspect of the invention may extract a clinical image that has been displayed by a user, from among multiple clinical images, and register the extracted clinical image as a key image.

A device for generating a key medical image according to still another aspect of the invention may extract a clinical image that has been displayed by a user for a particular duration of time, from among multiple clinical images, and register the extracted clinical image as a key image.

A device for automatically selecting a key image from medical images according to yet another aspect of the invention may select a clinical image that has undergone image processing, from among multiple clinical images, and register the selected clinical image as a key image.

The image processing can include adjusting an image size, adjusting an image brightness, adjusting an image color, and measuring an image distance.

A device for generating a key medical image according to another aspect of the invention may include: an input check module configured to identify a clinical image for which a record was inputted from among multiple clinical images; a display check module configured to identify a clinical image which has been displayed from among the multiple clinical images; a time check module configured to identify a clinical image which has been displayed for a particular duration of time or longer from among the multiple clinical images; and a key image registry module configured to assign points to clinical images identified by the input check module, the display check module, and the time check module and register a clinical image having a certain amount of points or more assigned thereto as a key image.

A device for automatically selecting a key medical image according to another aspect of the invention may include: an input check module configured to identify a clinical image for which a record was inputted from among multiple clinical images; a display check module configured to identify a clinical image which has been displayed from among the multiple clinical images; a time check module configured to identify a clinical image which has been displayed for a particular duration of time or longer from among the multiple clinical images; an image processing check module configured to identify a clinical image which has undergone image processing from among the multiple clinical images; and a key image registry module configured to assign points to clinical images identified by the input check module, the display check module, the time check module, and the image processing check module and register a clinical image having a certain amount of points or more assigned thereto as a key image.

### Advantageous Effects

An embodiment of the invention can automatically generate key images from among clinical images and thus obviate the need to transmit unnecessary clinical images.

Also, an embodiment of the invention can provide a device for automatically selecting key medical images that offers high reliability in choosing key images.

### BRIEF DESCRIPTION OF THE DRAWINGS

FIG. 1 is a diagram illustrating an example of the connections of a device for automatically selecting key images according to an embodiment of the invention.

FIG. 2 is a diagram illustrating an example of a device for automatically selecting key images according to an embodiment of the invention.

FIG. 3 is a diagram illustrating an example of key images selected by a device for automatically selecting key images according to an embodiment of the invention.

### DETAILED DESCRIPTION OF THE INVENTION

As the invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the description of the present invention, certain detailed explanations of the related art are omitted when it is deemed that they may unnecessarily obscure the essence of the present invention.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms such as “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Terms such as “first”, “second”, etc., can be used in describing various elements, but the elements are not to be limited by such terms. The terms are used only to differentiate one element from another.

Certain embodiments of the present invention are described below in more detail with reference to the accompanying drawings. In the description of the invention with reference to the accompanying drawings, the same or corresponding elements may be rendered the same reference numeral, regardless of the figure number, and redundant descriptions may be omitted.

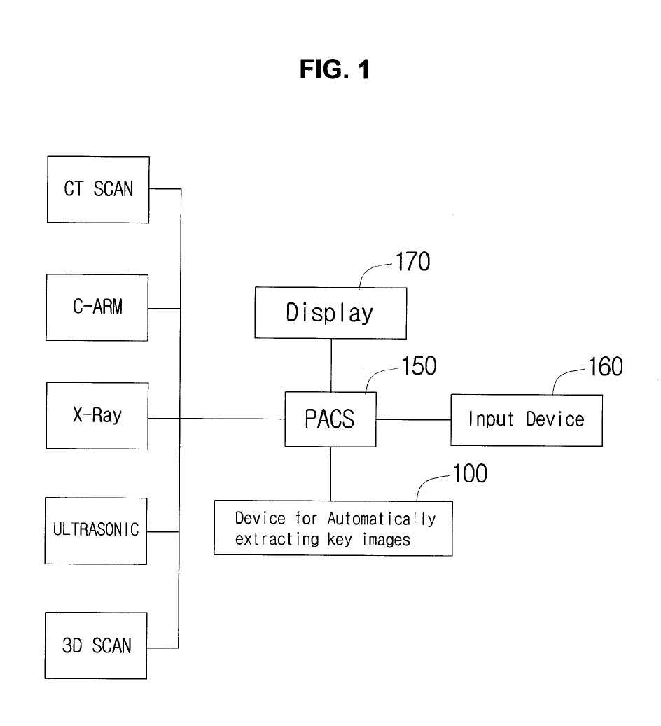

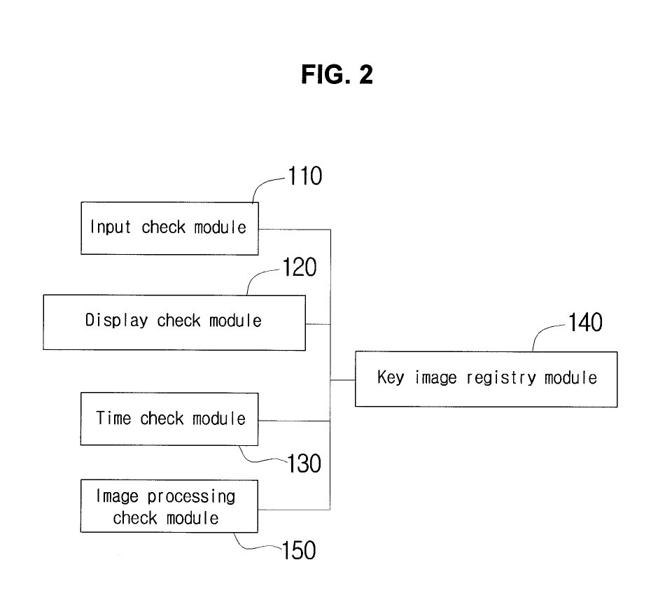

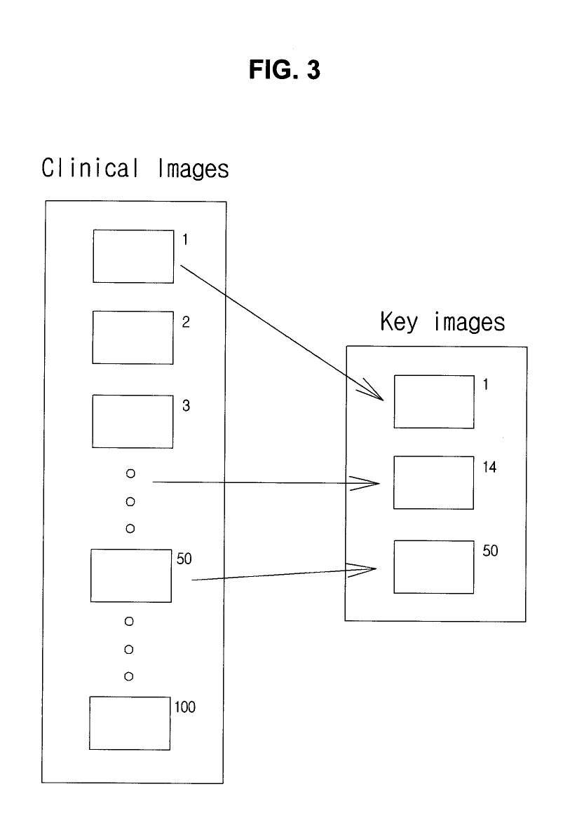

FIG. 1 is a diagram illustrating an example of the connections of a device 100 for automatically selecting key images according to an embodiment of the invention, and FIG. 2 is a diagram illustrating an example of a device 100 for automatically selecting key images according to an embodiment of the invention. FIG. 3 is a diagram illustrating an example of key images selected by a device 100 for automatically selecting key images according to an embodiment of the invention.

Referring to FIG. 1 through FIG. 3, a device 100 for automatically selecting key images according to this embodiment relates to a device for selecting clinical images that have unique qualities (for example, a clinical image in which a hematoma is observed) or require careful observation by an evaluator, from among many medical images, and automatically registering these images as key images.

Clinical images generated by a medical imaging device (modality) such as CT scan, C-arm, X-ray, ultrasonic, and 3D scan devices may be stored in a PACS 150 after they are converted into a DICOM file. The clinical images stored thus may be provided to the medical staff through a display 170 located within the hospital. The medical staff may view the clinical images on the display 170 and can input a finding, etc., into a clinical image by using an input device 160. The input device 160 can correspond to an EMR (electronic medical system) or a RIS (radiology information system).

A device 100 for automatically selecting key images according to this embodiment can be implemented as a separate device that is connected to a PACS 150, which is a system for storing and transferring medical images, or can be implemented as a module within the PACS 150 or the input device 160.

A device 100 for automatically selecting key images according to this embodiment may include an input check module 110, a display check module 120, a time check module 130, an image processing check module 150, and a key image registry module 140.

The input check module 110 may select clinical images for which users' records (for example, evaluation findings) have been inputted, from among the large number of clinical images. If a user inputs a record such as an evaluation finding on the input device 160 while a particular clinical image is being displayed, then the input check module 110 can consider said clinical image as a key image. The key image registry module 140 can then register the clinical images that have been selected by the input check module 110 (and have records by the medical staff inputted therein) as key images.

The display check module 120 may select clinical images that have been displayed by the user, from among the large number of clinical images. The display check module 120 may calculate the number of times a clinical image has been displayed by the user for each of the clinical images and may consider those clinical images that have been displayed a designated number of times or more as key images. An image may be determined as having been displayed whenever the image is displayed by the user regardless of the duration of the display or only when the image is displayed for a designated duration of time or longer. The key image registry module 140 can register the clinical images that have been selected by the display check module 120 as key images.

The time check module 130 may select clinical images that have been displayed by the user for a certain duration of time, from among the large number of clinical images. The time check module 130 may calculate the duration of time a clinical image has been displayed for each of the clinical images and may consider those clinical images that have been displayed for a designated duration of time or longer as key images. The display duration used can be the duration for one instance of displaying for each clinical image or the total cumulative duration of time regardless of the number of display instances. The key image registry module 140 can register the clinical images that have been selected by the time check module 130 as key images.

The image processing check module 150 may select clinical images that have undergone image processing from among the clinical images. The image processing can include, but is not limited to, adjusting an image size performed by a user such as by magnifying or shrinking the image, adjusting an image brightness, adjusting an image color, and measuring an image distance (measurement of distance for a particular portion within the PACS).

While the key image registry module 140 can register as key images the clinical images selected by the input check module 110, display check module 120, and time check module 130 individually, it is also possible to use a combination of the above images. For example, the key image registry module 140 can register those clinical images that have been commonly selected by the input check module 110 and the time check module 130 as key images.

The key image registry module 140 can assign points to clinical images selected by the input check module 110, display check module 120, time check module 130, and image processing check module 150 and can register those clinical images that have a certain amount of total assigned points or more as key images. For example, from among the clinical images, an image having an inputted record can be assigned 3 points, an image having no inputted record can be assigned 1 point, an image that was displayed twice or more can be assigned 5 points, an image that was displayed once can be assigned 3 points, and an image that was not displayed at all can be assigned 1 point. Similarly, an image that underwent image processing can be assigned 3 points, and an image that was not processed can be assigned 1 point. With points assigned in this manner, those clinical images having a total point of 7 points or more can be registered as key images. That is, if a particular clinical image has a record inputted therein, has been displayed once, and has been image processing, then its total assigned points would be 9 points, and the corresponding clinical image can be registered by the key image registry module 140 as a key image.

Also, images to which image processing has been applied can be assigned points differently. For example, an image can be assigned 5 points for a size measurement, 3 points for adjusting at least one of brightness and color, and 1 point for magnifying or shrinking the image.

The clinical images chosen as key images may be registered in the PACS 150 as a KOS (key object selection) and automatically inputted into a DICOM file. When clinical images are transmitted by way of a KOS file, it becomes possible to transmit only the clinical images that have registered as key images. As illustrated in FIG. 3, a device 100 for automatically selecting key images may select clinical images no. 1, 14, and 50, from among a hundred clinical images, and only the registered key images can be transmitted.

Since a device 100 for automatically selecting key images according to this embodiment may thus automatically select clinical images that have unique qualities or require careful observation by an evaluator and register these images as key images, the transmitting of unnecessary clinical images can be avoided. Also, since a device 100 for automatically selecting key images according to this embodiment may register images according to whether or not records (doctor's opinions) have been inputted, whether or not the images have been displayed, whether or not the images have been displayed for over a certain duration of time, and whether or not there was image processing, the selection of key images may be performed with high reliability.

While the present invention is described above using certain embodiments of the present invention, the person having ordinary skill in the art would understand that the invention can be modified and altered in various ways without departing from the spirit and scope of the present invention recited in the claims below.

What is claimed is:

1. A device for generating a key medical image, the device comprising:
an input check module configured to identify a clinical image having a record inputted therein by a user from among a plurality of clinical images; and
a key image registry module configured to extract a clinical image identified by the input check module and register the extracted clinical image as a key image.

2. A device for generating a key medical image, the device comprising:
a display check module configured to identify a clinical image which has been displayed by a user from among a plurality of clinical images; and
a key image registry module configured to extract a clinical image identified by the display check module and register the extracted clinical image as a key image.

3. A device for generating a key medical image, the device comprising:
a time check module configured to identify a clinical image which has been displayed by a user for a particular duration of time or longer from among a plurality of clinical images; and
a key image registry module configured to extract a clinical image identified by the time check module and register the extracted clinical image as a key image.

4. A device for automatically selecting a key medical image, the device configured to select a clinical image which has undergone image processing from among a plurality of clinical images and register the selected clinical image as a key image.

5. The device for automatically selecting a key medical image according to claim 4, wherein the image processing comprises adjusting an image size, adjusting an image brightness, adjusting an image color, and measuring an image distance.

6. A device for generating a key medical image, the device comprising:
an input check module configured to identify a clinical image having a record inputted therein from among a plurality of clinical images;
a display check module configured to identify a clinical image which has been displayed from among the plurality of clinical images;
a time check module configured to identify a clinical image which has been displayed for a particular duration of time or longer from among the plurality of clinical images; and
a key image registry module configured to assign points to clinical images identified by the input check module, the display check module, and the time check module and register a clinical image having a certain amount of points or more assigned thereto as a key image.

7. A device for automatically selecting a key medical image, the device comprising:
an input check module configured to identify a clinical image having a record inputted therein from among a plurality of clinical images;
a display check module configured to identify a clinical image which has been displayed from among the plurality of clinical images;
a time check module configured to identify a clinical image which has been displayed for a particular duration of time or longer from among the plurality of clinical images;
an image processing check module configured to identify a clinical image which has undergone image processing from among the plurality of clinical images; and
a key image registry module configured to assign points to clinical images identified by the input check module, the display check module, the time check module, and the image processing check module and register a clinical image having a certain amount of points or more assigned thereto as a key image.
